(19) United States
(12) Patent Application Publication
(10) Pub. No.: US 2004/0216175 A1
(43) Pub. Date: Oct. 28, 2004

(54) Detection and identification of toxicants by measurement of gene expression profile

(76) Inventors: Hyesook Kim, Bloomfield Hills, MI (US); Mary Murray, Grosse Pointe, MI (US); Raymond F. Novak, Orchard Lake, MI (US)

Correspondence Address: Amy E. Rinaldo, KOHN & ASSOCIATES, PLLC, Suite 410, 30500 Northwestern Highway, Farmington Hills, MI, 48334, US

(21) Appl. No.: 10/780,339

(22) Filed: Feb. 17, 2004

Related U.S. Application Data
(60) Provisional application No. 60/448,266, filed on Feb. 17, 2003.

(51) Int. Cl. C12Q 1/68; A01K 67/00

(52) U.S. Cl. 800/8; 435/4; 435/6

(57) ABSTRACT

A screen for detecting, identifying, and characterizing chemicals as toxicants is based on the affect of the chemical on gene expression in animal cleavage stage embryos. A microarray screen for detecting and measuring the affects of chemicals on gene expression in animal cleavage stage embryos. A screen for detecting, identifying, and characterizing chemicals as toxicants based on the common or differential affects on gene expression in animal cleavage stage embryos and neurulation stage embryos. Markers of chemical exposure and teratogenesis identified using the screen disclosed herein. A treatment that enables the transfer of biotinylated PCR products or DNA to a membrane following gel electrophoresis by depurinating the PCR or DNA products and denaturing the PCR products or DNA.

### CROSS-REFERENCE TO RELATED APPLICATIONS

The present application claims the benefit of priority under 35 U.S.C. Section 119(e) of U.S. Provisional Patent Application No. 60/448,266, filed Feb. 17, 2003, which is incorporated herein by reference.

### GOVERNMENT SUPPORT

Research in the application was supported in part by a contract from National Institute of Environmental Health Sciences (ES 15462). The government has certain rights in the invention.

### BACKGROUND OF THE INVENTION

1. Technical Field

Generally, the present invention relates to a method and screen for detecting and identifying toxins using animal cleavage stage embryos. More specifically, the present invention provides a method and screen for detecting and identifying chemicals that affect gene expression as an indicator of toxicity. This invention relates to a screen to identify chemicals as toxicants by detecting patterns of altered gene expression induced by chemical treatment of cleavage stage animal embryos using various techniques including microarray analysis. More specifically the present invention relates to a screen that can be used to identify chemicals as toxicants using animal embryos during the earliest period of development, the cleavage stage, when the first differentiation of cell type occurs. The present invention also relates to a screen to identify chemicals as toxicants by comparing the affect of the chemical on gene expression in animal embryos undergoing cleavage and neurulation. The present invention also relates to the use of genes identified as highly up-regulated or highly down-regulated in animal cleavage stage embryos by chemical treatment as markers of chemical exposure. More specifically the present invention relates to the use of genes identified as highly up-regulated or down-regulated in chemically-treated animal cleavage stage embryos as markers of tertatogenesis where chemical treatment blocked embryonic differentiation. The present invention also relates to treatment of biotinylated DNA by depurination and denaturation to enable efficient transfer of DNA to a membrane following gel electrophoresis.

2. Description of Related Art

Animal experiments have been carried out under the assumption that results obtained with the study can be extrapolated to human study. Animal experiments are commonly used to screen toxic effects of drugs and chemicals used in the household and industry and pesticides used for farming.

Xenopus laevis provides a well-established model of embryo development that can be used for analysis of chemical exposure. Inter-laboratory studies demonstrated the Frog Embryo Teratogenesis Assay—Xenopus (FETAX) using late blastula stage Xenopus laevis embryos in a 96 hour whole-embryo assay is reliable and predictive for toxicity and teratogenicity (1,2). It is also useful for screening environmental samples of complex mixtures. FETAX evaluates survival, malformation, ability to swim, skin pigmentation, stage of development, and growth. The method measures the LC50 (the 96 hour median lethal concentration) and EC50 (the concentration inducing malformation in 50% of the surviving embryos) of toxicants (1,2). However, FETAX does not apply or enable any molecular or biochemical analysis.

Embryogenesis initiates upon fertilization of the egg with the first cell division. The early period of embryogenesis in all animals is a cleavage stage characterized by repeated cell divisions without growth resulting in progressively smaller cells in the embryo. Early embryogenesis depends initially on maternally inherited molecules and structures that are gradually replaced by ones synthesized in the embryo. Onset of transcription from the embryo genome varies between species. In all embryos the initial cleavage stage depends on maternally inherited components. In Xenopus, the entire period of cleavage stage depends on maternally inherited components, with the onset of embryonic transcription coinciding with the onset of gastrulation at the mid-blastula transition (3,4). In Xenopus, maternally inherited mRNAs that are layed down in the oocyte in an inactive form are recruited for protein synthesis during the cleavage stage. In addition to the large store of maternal mRNA that is recruited during cleavage, selective transcription contributes small amounts of embryonic mRNA (5).

More specifically, Xenopus embryos provide a facile system for investigating embryogenesis. In all animal embyros, one of the first differentiation events is the formation of ectoderm, endoderm and mesoderm cell lineages called the germ layers. Subsequently, gastrulation transforms the spherical blastula embryo into a structure with a hole through the middle that becomes the gut. In Xenopus, the germ layers are formed in the blastula, stages 8.5-9, and gastrulation begins in the early gastrula at stage 10. Recently, microarray analysis of gene expression in early Xenopus laevis development was reported using microarrays composed of Xenopus laevis gastrula cDNAs (11). Three investigations were pursued in the study: 1) comparison of maternal versus gastrula transcription, 2) spatially restricted gene expression in the gastrula embryo and 3) induction of mesoderm germ cells at midgastrula using isolated blastula ectoderm cells treated with the Xenopus laevis protein growth factor activin, a known inducer of mesoderm differentiation. Each of these observations provided confirmation of previously known outcomes determined with other molecular technologies. No part of this study involved cleavage stage embryos. The paper concludes with the statement, “based on the success of the prototype arrays, the larger scale arrays should allow the rapid identification of regulated genes under a variety of conditions (page 74).” However, it is important to note that the study was limited to investigating the events of normal embryonic development. Moreover, there is no mention of investigating the impact of chemical treatment on embryogenesis.

An expressed sequence tag (EST) is a nucleotide sequence obtained from a cDNA insert by single-run sequencing. Usually, an EST is a short (˜300-500 bp) 5′- or 3′-end cDNA sequence that includes a coding or non-coding region. Since Adams et al. (6) pioneered the collection of ESTs to be used for gene mapping, new gene discovery, and identification of coding regions in genomic sequences, the utility of ESTs has been widely investigated for many organisms including Xenopus laevis. With the advent of EST sequencing projects, the UniGene system for partitioning GenBank sequences into non-redundant gene clusters was initiated. As of Dec. 12, 2003, the UniGene Build #48 identified 276,122 Xenopus laevis sequences in clusters representing 21,810 unique genes.

Regulation of expression of genes with a known or unknown function has been analyzed by a throughput method such as microarray technology that simultaneously monitors expression of thousands of genes (7-9). The technology has emerged as a primary tool for Molecular Toxicology (10). Automation of microarray chip construction, use of fluorescent signals and custom digital image analysis makes it possible to monitor gene expression of thousands of genes and obtain expression profiles of environmental toxicants (10).

Base pairing (i.e., A-T and G-C for DNA; A-U and G-C for RNA) or hybridization is the underlining principle of DNA microarray. An array is an orderly arrangement of samples. It provides a medium for matching known and unknown DNA samples based on base-pairing rules and automating the process of identifying the unknowns. An array experiment can make use of common assay systems such as microplates or standard blotting membranes, and can be created by hand or through the use robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarrays are typically less than 200 microns in diameter and the arrays usually contain thousands of spots.

Microarrays require specialized robotics and imaging equipment that generally are not commercially available as a complete system. DNA microarray, or DNA chips are fabricated by high-speed robotics, generally on glass but sometimes on nylon substrates, for which probes* of defined character are used to determine complementary binding, thus allowing massively parallel gene expression and gene discovery studies. An experiment with a single DNA chip can provide researchers information on thousands of genes simultaneously, a dramatic increase in throughput.

Micrroarray biochips are being increasingly used for the performance of large numbers of closely related chemical tests. For example, to ascertain the genetic differences between lung tumors and normal lung tissue one might deposit small samples of different cDNA sequences under a microscope slide and chemically bond them to the glass. Ten thousand or more such samples can easily be arrayed as dots on a single microscope slide using mechanical microarraying techniques. Next, sample mRNA is extracted from normal lung tissue and from a lung tumor. The mRNA represents all of the genes expressed in the tissues and the differences in the expression of mRNA between the diseased tissue and the normal tissue can provide insights into the cause of the cancer and perhaps point to possible therapeutic agents as well. The “probe” samples from the two tissues are labeled with different fluorescent dyes. A predetermined amount of each of the two samples is then deposited on each of the microarray dots where they competitively react with the cDNA molecules. The mRNA molecules that correspond to the cDNA strands in the array dots bind to the strands and those that do not are washed away.

The slide is subsequently processed in a scanner that illuminates each of the dots with laser beams whose wavelengths correspond to the fluorescence of the labeling dyes. The fluorescent emissions are sensed and their intensity measured to ascertain for each of the array dots the degree to which the mRNA samples correspond to the respective cDNA sequences. In the experiment outlined above, the image scanner separately senses the fluorescence and thereby provides separate maps of the reactions of the mRNA extracted from the normal and tumorous tissues. The scanner generates an image map of the array, one for each of the fluorescenses. The maps are ultimately analyzed to provide meaningful information to the experimenter.

Microarray biochips are available in a variety of factors and can contain one or more different fluorescence labels. The reagents involved in the chemical reactions in the array dots are typically biological samples such as DNA, RNA, peptides, proteins or other organic molecules. The biochips might be used for diagnostics, screening assays, genetics and molecular biology research. They can include, in addition to the test dots, calibration dots containing known amounts of the fluorescent materials. Scanning of the latter dots thus serves to calibrate the readings obtained from the test dots.

It would therefore be useful to develop microarrays for determining and investigating toxicity in early animal embryos. There is therefore a need for a method of detection and classification of toxicants by measurement of up or down-regulated gene expression. Accordingly, toxicity in animal cleavage stage and neurulation stage embryos could be used to detect and identify various toxins that affect gene expression in any biological system.

### SUMMARY OF THE INVENTION

According to the present invention there is provided a screen for detecting, identifying, and characterizing chemicals as toxicants based on the affect of the chemical on gene expression in animal cleavage stage embryos. Also provided is a microarray screen for detecting and measuring the affects of chemicals on gene expression in animal cleavage stage embryos. Markers of chemical exposure and teratogenesis identified using the screen disclosed herein are also provided. A treatment enabling the transfer of biotinylated PCR products or DNA to a membrane following gel electrophoresis by depurinating the PCR or DNA products and denaturing the PCR products or DNA is provided.

### DESCRIPTION OF THE DRAWINGS

Other advantages of the present invention will be readily appreciated as the same becomes better understood by reference to the following detailed description when considered in connection with the accompanying drawings wherein:

FIGS. 1A-C are images showing representative pseudo-colored microarray analysis of Xenopus laevis cleavage and neurulation stage embryos treated with PMA to identify patterns of altered gene expression;

FIG. 2 is an image of microarray analysis showing the application of the Xenopus laevis microarray to Rana pipiens gene expression, the Xenopus laevis microarray was probed with Rana pipiens liver mRNA [labeled Cy5 (red)]; and

FIGS. 3A-C are images of RT-PCR products obtained using biotinylated primers of clone No. PBX0135A08 to quantitate expression of the gene corresponding to PBX0135A08 in Xenopus laevis embryos.

### DETAILED DESCRIPTION OF THE INVENTION

Generally, the present invention provides a method of detecting and identifying chemicals that alter a gene expression profile within a biological system. Additionally, the present invention provides markers of chemical exposure and teratogenesis, the markers being identified by the method and screen of the present invention.

The term “screen” as used herein can include any device capable ot screening for gene expression in an embryo. An example of such a screen includes, but is not limited to, a microarray.

The term “chemical” as used herein can include any chemical suspected of affecting gene expression. Examples of such chemicals include, but are not limited to, inducers of cellular proliferation and other gene expression modifying compounds. An specific inducer of cellular proliferation is phorbol ester, preferably phorbol 12-myristate 12-acetate (PMA).

The term “embryo” as used herein can include any animal embryo. Examples of embryos that can be used include, but are not limited to, vertebrate animals including and aquatic species and amphibians such as Xenopus and specifically Xenopus laevis and other embryos known to those of skill in the art to be effective in the screen of the present invention.

The term “modulation” as used herein is intended to include both up regulation and down regulation. In other words, the method of the present invention can be used to detection of both up regulated genes and down regulated genes.

The term “marker” as used herein is intended to include genes whose expression is indicative of chemical exposure or teratogenesis, the development of malformations or serious deviations from the norm in organisms. Examples of such genes are included in Table 1 through Table 5, and homologs thereof.

The present invention provides for the detection and classification of toxicants by the measurement of up or down-regulated gene expression using molecular toxicology tools. The method detects patterns of altered gene expression induced by chemical treatment of cleavage stage animal embryos using various techniques including microarray analysis. More specifically, the present invention provides mRNA expression analyses of frog embryos after treatment with xenobiotics including toxicants and food additives to facilitate investigations of physiologic and pathologic roles of genes.

The screen and method of the present invention identify chemically-induced patterns of altered gene expression by measuring the effects of chemical treatment on gene expression in animal cleavage stage embryos is disclosed. Cleavage stage is the earliest embryonic stage depending on gene products expressed from the maternal genome inherited from the egg. The cleavage stage is characterized by cell division without cell growth. The unexpected finding was that gene regulation at cleavage stages was extremely sensitive to chemical treatment. Considering that embryogenesis is highly conserved among animals, gene regulation studies for animals after chemical treatment of embryos, especially Xenopus and mice, at the early embryonic stages have advantages of shorter incubation time after fertilization of the embryos and higher sensitivity. Studying gene expression in embryos is disclosed in the prior art, for example using methods such as FETAX. The method of the present invention differs from FETAX, because FETAX uses Xenopus laevis in a 96-hour assay for the evaluation of physical malformations. FETAX uses Xenopus laevis late stage blastula embryos (stage 9) in a 96 hour assay with physical malformations evaluated in the end stage 41 embryo (1,2). Cleavage stage is completed in the stage 8 embryo and therefore is not included in FETAX. Thus, the method of the present invention analyzes gene expression at a much earlier stage than previously thought possible and is able to identify genes that are up or down regulated during cleavage.

The genes that are identified as highly up-regulated or down-regulated in the cleavage stage embryo by PMA-treatment as markers of chemical treatment and teratogenesis. The genes can be used as markers of tertatogenesis since PMA-treatment blocked embryonic differentiation. More specifically, the present invention provides a method that can be used to identify genes that are up-regulated or down-regulated as a result of chemical treatment of animal embryos during the earliest period of development, the cleavage stage, when the first differentiation of cell type occurs. The method can identify genes that are uniquely up regulated or down regulated by chemical treatment of animal embryos during cleavage or neurulation. The method can be used to identify genes that are differentially responsive to chemical treatment of animal embryos during cleavage stage or during neurulation.

The present invention utilizes the Xenopus laevis species because it is a facile model to investigate developmental toxicity. X. laevis microarray analysis is a versatile tool for drug screening and mechanistic studies of environmental toxicology. Microarray technology is utilized on a glass chip printed with PCR products of Xenopus expressed sequence tag (EST) clones.

The present invention provides for improved X. laevis microarray technology, categorization of a few typical environmental toxicants according to their gene expression profiles, and discovery of sensitive markers of environmental contaminants.

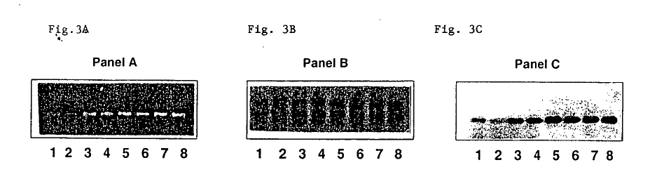

The present invention also uses depurination and denaturation as a method to enable transfer of biotinylated DNA to a membrane following gel electrophoresis. Quantitation of DNA species often employs transfer of DNA separated by gel electrophoresis to membrane supports upon which the quantitative assay is carried out. Synthesis of cDNA using a biotin-labeled primer in reverse transcription coupled PCR (RT-PCR) assay allows quantitation of the biotin-labeled cDNA using horseradish peroxidase coupled ECL. ECL provides a sensitive method for DNA quantitation however it depends on efficient transfer of the biotin-labeled DNA to a membrane support upon which ECL is carried out. The unexpected observation that a 380 bp biotin-labeled cDNA did not efficiently transfer from an agarose gel to a membrane resulted in inaccurate quantitation of the 380 bp biotin-labeled cDNA (FIGS. 3A and B). Specifically, the major 380 bp species was detected in similar quantity as a minor ˜400 bp species (FIGS. 3A and B). Denaturation and depurination of the gel allowed efficient transfer of the major biotin-labeled 380 bp species (FIG. 3C) so that the DNA could be accurately quantitated. Denaturation and depurination of biotin-labeled DNA contained in a gel matrix prior to transfer to a support membrane represents a critical improvement in a process that is widely used.

Generally, the method of the present invention includes the steps of treating embryos, for example Xenopus laevis at stages 8 (blastula) and 15 (neurula), with a chemical such as PMA and analyzing the effects of the chemical on morphology and gene expression. The method of the present invention can identify chemically induced patterns of altered gene expression by measuring the effects of chemical treatment on gene expression in animal cleavage and neurulation stage embryos.

Specifically, the method and screen of the present invention function as follows. EST clones produced from Xenopus laevis unfertilized eggs were used in the present study with ˜1,200 EST clones from a 18,500 EST clone set selected for production of a Xenopus cDNA microarray. The Xenopus microarray was used to measure the effect of chemical treatment on Xenopus embryo gene regulation. The chemical treatment was a phorbol ester, PMA. Xenopus eggs were obtained from females induced for ovulation and fertilized in vitro. Control Xenopus laevis embryos were allowed to develop to stage 8 blastula or stage 15 neurula. Treated embryos were exposed to PMA during cleavage stage or during neurulation with end points of blastula (stage 8) or neurula (stage 15). Specifically, PMA 100 ng/ml was added to the incubation media during cleavage stages for evaluation in stage 8 embryo or PMA was added during neural induction for evaluation in stage 15 embryos. RNA extracted from the embryos was used to generate fluorescent cDNA probes for Xenopus microarray analyses to measure differential gene expression with and without the phorbol ester treatment. Analysis was performed using paired RNA samples to compare PMA-treated and untreated embryos: Group I measured the effects of PMA-treatment during the cleavage stage in stage 8 embryos and Group II measured the effects of PMA-treatment during neurulation in stage 15 embryos. Group III measured the change in gene expression between stage 8 and stage 15 embryos.

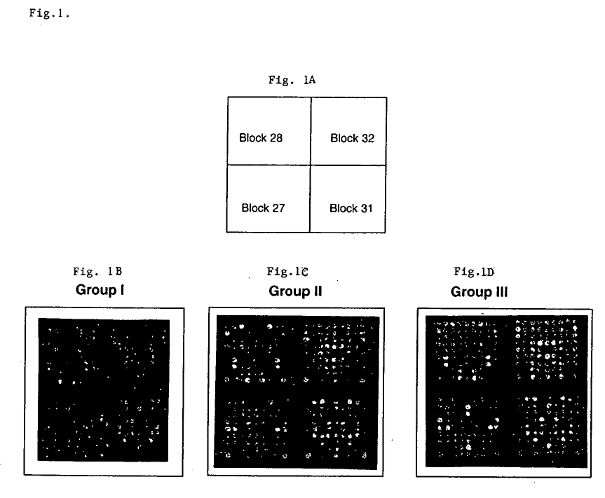

Representative microarray images for Group I, II and III are presented in FIG. 1 with data from presented in Tables 1A, 3-5. Xenopus genes that are highly up-regulated or down-regulated in cleavage stage embryos (Table 1A and 3) as a result of PMA-treatment are claimed as markers of PMA-treatment.

Xenopus eggs contain a mass store of RNA transcribed during oogenesis that is used as the primary source of RNA during cleavage stages since general transcriptional activation in the embryo does not occur until the midblastula transition at embryonic stage 8.5 (3-5). The chemical treatment was thought to only significantly effect gene expression after the midblastula transition (stage 8.5) when embryonic transcription is activated, and that there would be little or no effect on gene expression in the cleavage stage embryo. It was anticipated that PMA-treatment of cleavage stage embryos would have little effect on the pattern of gene expression. However contrary to the prediction, the results were surprising in that many up- or down-regulated genes were identified after PMA-treatment of cleavage stage embryos (Table 1 and 3).

It was anticipated that treatment of embryos during neurulation would result in significant changes in gene expression. PMA-treatment during neurulation resulted in up-regulation of some genes (Table 4) but a greater effect on down-regulation was observed in the stage 15 embryo (Table 5). Comparison of genes highly up-regulated or down-regulated by PMA-treatment of cleavage stage and neurula stage embryos revealed differential regulation of Xenopus genes by PMA in the different stages. Specifically, 7 of the 25 genes that are highly up-regulated, and 1 of the 24 genes that are highly down-regulated in the cleavage stage embryo by PMA-treatment are similarly up- or down-regulated by PMA-treatment of neurulation stage embryos. Measurement of the effect of chemicals on gene expression using embyros at both cleavage and neurulation stages can determine common or differential effects of chemicals on gene expression of embryos.

Verification of microarray analysis by RT-PCR was performed for selected genes up-regulated by PMA-treatment during the cleavage stage (Table 2; FIG. 3). Results from microarray analysis and RT-PCR were in agreement. The genes represented by ESTs PBX0135A08 and PBX013409 were highly up-regulated by PMA-treatment of Xenopus cleavage embryos (Table 2 stage 8) but were unaffected by PMA-treatment of embryos undergoing neurulation (Table 2 stage 15).

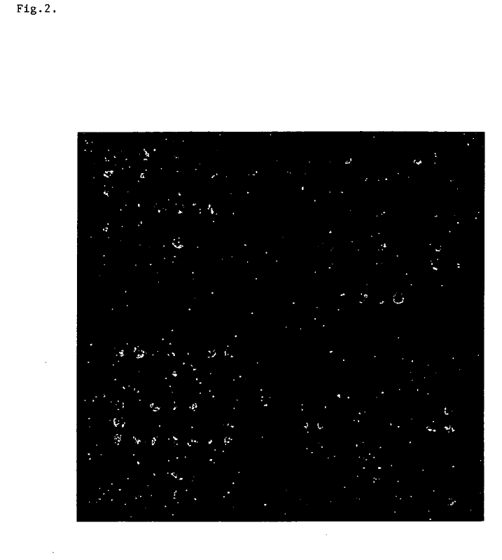

RNA from the North American frog Rana pipiens was used to probe the Xenopus microarray to evaluate application of the Xenopus microarray to other species. Substantial signal was selectively retained on the Xenopus microarray with the probe made from Rana pipiens liver RNA (FIG. 2) demonstrating utility of the Xenopus microarray for other frog species.

The invention is further described in detail by reference to the following experimental examples. The examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

### MATERIALS AND METHODS

Microarray Construction

cDNA fragments were obtained by PCR replication of Xenopus laevis unfertilized egg cDNA inserts, purified, quantitated and loaded onto glass chips by robotics. In addition to the selected ˜1,200 genes, 64 lambda Q gene, an internal control cDNA (Genomic Solutions, Inc., Ann Arbor, Mich.) was loaded on the chip. The insert DNA from ˜1,200 clones were PCR amplified using forward and reverse primers (GF.200 primers, Research Genetics, Huntsville, Ala.). A second PCR was carried out using 1 μl of the primary PCR (20 μl) for template. Excess dNTPs, polymerase and other PCR artifacts were removed from the secondary PCR reactions using Millipore Multiscreen plates (Millipore Corporation, Bedford, Mass.) using the standard protocol. The purity of the PCR products was assessed by electrophoresis and documented using an Alphalmager 1000 Digital Imaging System (Alpha Innotech Corporation, San Leandro, Calif.). The DNA obtained by PCR was quantitated using PicoGreen (Molecular Probes, Eugene, Oreg.). The DNA was denatured and arrayed onto amino-silane coated microscope slides (CMT-GAPS coated slides, Corning, Corning, N.Y.) using a Flexys robotic workstation (Genomic Solutions Inc.). The ˜1,200 PCR products were arrayed in duplicate on each slide and cross-linked. Each gene chip contained duplicate cDNA samples.

Preparation of Fluorescent Probes and Hybridization.

Total RNA was prepared from embryos treated in three separate dishes per treatment group that were pooled. Total RNA was isolated from the frog embryos (1,200 embryos for each probe production) using TRIzol extraction (Gibco BRL, Grand Island, N.Y.) and Qiagen RNeasy kits (Qiagen, Valencia, Calif.). The ethanol precipitated RNA was resuspended in RNase-free water, quantitated with RiboGreen (Molecular Probes, Eugene, Oreg.). Total RNA was further purified using oligoTex mRNA maxi kit (Qiagen Co.) with the bound mRNA eluted with minimum volume of elution buffer and precipitated with ethanol. The recovery rate of mRNA by the ethanol precipitation method was ˜70%. Cy3- or Cy5-tagged probes were produced from an mRNA mixture (20 μl) of Xenopus mRNA (5 μg) and lambda Q gene mRNA (3.5 ng) reverse-transcribed with oligo(dT)₁₈₋₂₂ primer and dNTPs and the reverse-transcribed cDNA was cross-linked with Cy3 or Cy5 as described in Clontech Atlas™ Glass Fluorescent Labeling kit manual (www.clontech.com). After hydrolysis of the RNA and purification of the probe (Centricon 50, Millipore, Bedford, Mass.), the cDNA in TE buffer was quantitated by absorbance at 260 nm. The probes were stored at −20° C. and protected from light until used.

Probes prepared from RNA extracted from Xenopus laevis embryos were labeled with different fluorescence, Cy3 or Cy5, and hybridized to cDNAs printed on the chip with a mixture of the probes. The approach eliminates a normalization step between the images as would be required for adjustment of the differential labeling and detection with the two different fluors because of variation of amount of cDNA printed on each chip. Differential gene expression was obtained after PMA-treatment and non-treatment of cleavage stage embryos harvested at stage 8 (Group I), PMA-treatment and non-treatment of neurulation stage embryos harvested at stage 15 (Group II), and between developmental stages 8 and 15 (Group III).

Group I: Effects of PMA treatment at cleavage stage (stage 8): a mixture of no treatment/Cy3 and treatment/Cy5.

Group II: Effects of PMA treatment at neurulation stage (stage 15): a mixture of no treatment/Cy3 and treatment/Cy5.

Group III: Differential gene expression between cleavage stage (stage 8) and neurulation stage (stage 15): a mixture of no treatment/Stage 8/Cy3 and no treatment/Stage 15/Cy5.

Hybridization of the Probes with Microarray Chips:

The two fluorescent-labeled cDNA probe solutions (Cy3-labeled, green and Cy5-labeled, red, 750 ng each) were mixed, denatured and hybridized overnight at 50° C. The microarrays were then washed successively in 0.5×SSC/0.01% SDS, 0.05×SSC/0.01% SDS, 0.05×SSC/0.01% SDS, 70% ethanol, and 100% ethanol at a constant temperature of 25° C. Hybridization of the probes and washing the microarrays were accomplished using a GeneTAC Hybridization Station (Genomic Solutions). Hybridization was performed with an initial 10-minute denaturation at 75° C., probe insertion at 65° C. and hybridization stepped down from 65° C. for 3 hours, 55° C. for 3 hours to 50° C. for 10 hours. Slides were washed on the station at varying stringencies starting at 50° C. to room temperature. After hybridization, microarray images were obtained in a gray scale by scanning the chips at 532 nm (for green-tagged) or 635 nm (for red-tagged) and the gray scale images were false-colored in green and red, respectively. The pseudo-colored images were combined to produce microarray composite images. In the composite image, when equal amount of Cy-3 and Cy5-tagged probes were bound, the color of the spot is yellow. Imaging was carried out using GeneTAC Biochip Analyzer (Genomic Solutions) or GenePix 4000A (Axon Instruments, Inc., Foster City, Calif.). The Xenopus chip has 1152 genes spotted in duplicate in a 9×9 patch, 32 grid (block) array. Bacteriophage lambda Q-gene spotted as a positive marker at the A1 and I1 positions (left and right corners on the bottom of each block) of the 32 patches (blocks). Spotting occurs in a mirror pattern using a middle vertical line as the axis. For the middle vertical line, spotting occurs in a mirror pattern using the middle empty spot as the axis. Representative pseudo-colored microarray grid images obtained from Group I, II and III are shown in FIG. 1. The images were obtained without correction of the gray scale images to compensate for differences in labeling efficiency of Cy3 and Cy5. Group I was too green (Cy3) that can be corrected by multiplication by normalization factor (NF) higher than 1. Groups II and III were too red (Cy5) that can be corrected by NF lower than 1.

In Group I, embryos were Stage 8 blastula that were either untreated during cleavage stage [labeled with Cy3 (green)] or PMA-treated during cleavage stage [labeled with Cy5 (red)]. In Group II, embryos were Stage 15 neurula that were either untreated during neurulation [labeled with Cy3 (green)] or PMA-treated during neurulation [labeled with Cy5 (red)]. In Group III, embryos were untreated blastula [stage 8 labeled with Cy3 (green)] or untreated neurula [stage 15 labeled with Cy5 (red)]. A scheme of the microarray area shown in the image for Group I, Group II and Group III is depicted at the left.

Quantitative Analysis of Microarrays:

Quantitative analysis of the DNA microarrays was carried out using GeneTAC Genomic Integrator (version 2.5) or GenePix pro (version 3.0) software. In the analysis, both median and mean values of each spot (pixel size, 20) were calculated. However, median values were used for analysis of the data because the median is much less likely to be influenced by a few bad readings. The method minimizes the effect of any aberrant samples that could distort the population distribution. Ratios of median were calculated for each spot by dividing the spot volume (integrated intensity minus background) of the Cy5 channel (red, 635 nm) by the spot volume of the Cy3 (green, 532 nm). Normalization factor (NF) was calculated for each experiment by two different methods: (a) using all the data points (˜2,300) obtained by quantitation of the chip, and (b) using 64 landmark lambda Q-gene spots. Normalization of the ratio of median is necessary to correct for differences in labeling efficiency between probes. NF calculation from all data points assumes that total Cy5 signal is equal to total Cy3 signal. The primary advantage of using NF calculated from all data points is that a few erratic data points do not influence the outcome of the calculation. NF by landmarks assumes that total Cy5 signal of landmarks is equal to total Cy3 signal of the landmarks because the same amount of landmark mRNA is mixed with sample mRNA prior to probe production. A value higher than 1 means too much Cy3 or green and a value lower than 1 means too much Cy5 or red. Trends of NF values obtained for each experiment obtained by two different methods were in agreement.

Fertilization and Culture of Embryos:

Nine female adult (oocytes positive) African clawed frogs were obtained from Xenopus One, Inc. (Ann Arbor, Mich.). Ovulation was induced with double treatments (first treatment, 200 units and, after 5 hours, second treatment, 500 units) with human chorionic gonadotropin (Sigma Co.) and eggs were harvested by squeezing. Eggs from the frogs were pooled. The pooled eggs were fertilized in vitro and dejellied. Embryo cultures were carried out and staged according to the Normal Table (12). Mesoderm induction begins 3 hours after fertilization at 23° C. at Stage 6 (Morula stage; 48 blastomeres); neurula induction begins 10 hours after fertilization at 23° C.

PMA Treatment:

Embryos were sorted for successful cleavage to 2 cells (1.5 hours after fertilization), split into groups for subsequent treatment (triplicate treatments per each experimental group) and visually monitored for normal embryonic morphology. Rates of abnormal morphology were recorded for each group. Embryos at stage 8 were obtained after incubation of the fertilized embryos at 23° C. for 5 hours. PMA (100 ng/ml) or DMSO (0.01%, solvent used to dissolve PMA) was added to the embryos 2 hours after fertilization. Whereas embryos without PMA treatment progressed to blastula stage, PMA-treated embryos remained in a pre-blastula stage, suggesting that PMA treatment impaired differentiation. Embryos at stage 15 were obtained after incubation of the fertilized embryos at 18° C. for 30 hours (the condition produced stage 15 embryos identical to embryos obtained at 23° C. for 10 hours). PMA or DMSO was added to the embryos 21 hours after fertilization. Whereas embryos without PMA treatment showed typical morphology of neurula, PMA-treated embryos were in a pre-neurula stage. Embryos treated with 4 alpha-PMA, a stereoisomer of PMA that is ineffective at activating protein kinase C, showed typical neurula morphology.

Verification of Microarray Analysis by RT-PCR:

PCR primers sequences of the PMA up-regulated genes shown in Table 1, Panel B, except for gene No. 3 (PBX0143E06), were selected and biotinylated primers were obtained from Invitrogen Life Technology (Grand Island, N.Y.). Up-regulation of the clone Nos. PBX0135A08 and PBX0134E09 (underlined and shaded genes in Table 1, Panel A) were verified by RT-PCR of the genes. As predicted by microarray analyses, both genes were up-regulated after PMA treatment at stage 8 but not in stage 15. The DNA product obtained by RT-PCR using biotinylated primers of clone No. PBX0135A08 was separated by 1% agarose gel electophoresis and visualized by ethidium bromide staining (FIG. 3, Panel A). The DNA fragments were blotted to a nitrocellulose membrane and visualized by a streptavidin-horseradish peroxidase/ECL system (FIG. 3, Panel B). Though a single band for each reaction was visible in the ethidium bromide stained gel (FIG. 3, Panel A), multiple bands were obtained by the ECL system (FIG. 3, Panel B). When the biotin-labeled DNA in the gel was depurinated and denaturated by treatment with 0.25 M HCl followed by 1.5 M NaCl/0.56 N NaOH, the major 380 bp species were efficiently transferred and similar pattern to that observed using the ECL method was obtained (FIG. 3, Panel C). The effect was also observed with RT-PCR of clone No. PBX0134E09. mRNA levels of clones No. PBX0135A08 and No. PBX013409 increased after PMA treatment at stage 8 (Table 2).

In FIG. 3 the DNA products were obtained from 2 independent RT-PCR reactions using mRNA from untreated blastula stage 8 embryos (lanes 1 and 2), PMA-treated cleavage stage 8 embryos (lanes 3 and 4), untreated neurula stage 15 embryos (lanes 5 and 6), and PMA-treated neurula stage 15 embryos (lane 7 and 8). The 380 bp biotin-labeled DNA fragments were separated by gel electrophoresis on 1% agarose gel without further treatment (panel A and B) or following depurination and denaturation (panel C). In panel A, the DNA was detected by ultraviolet light following ethidium bromide staining. In panels B and C, the DNA was transferred to nitrocellulose membrane and detected by streptavidin-horseradish peroxidase ECL.

Application of Xenopus laevis Microarray to Rana pipiens:

Cy5-tagged (red) probe from Rana pipiens liver mRNA (5 μg) was prepared according to the method used for Xenopus mRNA. The Cy5-probe was denatured and hybridized to the microarray at 65° C. for 3 hours, 55° C. for 3 hours, and 50° C. for 10 hours using a Tecan hybridization station. The microarray was washed successively with medium agitation in 0.5×SSC/0.01% SDS 50C; 0.5×SSC/0.01% SDS 25° C. and 0.5×SSC 25° C. A close up area of the microarray is shown in FIG. 2 with the red signal derived from Cy5-Rana pipiens probe bound to the Xenopus laevis ESTs.

### Example 1

Identification of Genes that are Highly Up- or Down Regulated by PMA-Treatment of Cleavage Stage Embryos.

Genes that are highly up-regulated (Table 1, Panel A) or down-regulated (Table 3) after PMA treatment of Xenopus cleavage stage embryos harvested at stage 8 were identified. Duplicate ratios of the median for two data points and mean values were obtained. The mean values were multiplied by the NF obtained by the two different methods disclosed above. The final fluorescence ratios (differential expression) were an average of the ratios of the two independent hybridizations. The mean of the two values was obtained and multiplied by the NF. The up- or down-regulated genes were identified on images and their colors were visually confirmed. The levels of expression of each gene varied, i.e. PBX0135A08 was low. However, the data points showed “Flags” as “0” indicating that the data can be used for data mining. When the data point is not correct, i.e., an empty spot, the data sheet shows a minus value. “Flags” for the empty spot was −75.

Sequences of genes up-regulated by PMA treatment were obtained from GenBank using their clone ID's. A cDNA sequence size larger than 500 bp without any erratic sequences, such as repeated stretches of one nucleotide after another, was selected. The nucleotide sequence of the selected gene was cut and pasted in the BLASTN search window and matching sequences in GenBank were retrieved (Table 1, Panel B). cDNA sequences from clone ID PBX0141G10 and PBX0145H10, up-regulated genes after PMA treatment, have high % identity with a few Xenopus cDNA sequences in GenBank but the size of the homologous sequences was limited to 60-300 bp out of the total length ˜600 bp. Both of the cDNAs, which have entirely different cDNA sequence, have high % identity with two different segments of Xenopus aldolase gene (bold type in Table 1, Panel B). cDNA sequences from clone ID PBX0135A08 did not match with any Xenopus sequences reported in GenBank but a segment of the cDNA matched with chicken CD9 antigen and human antigen similar to CD9 antigen (Table 1, Panel B). cDNA sequences from clone ID, PBX0134E09, PBX0137G06 and PBX0136B06, PMA up-regulated genes, did not match with any GenBank entries (Table 1, Panel B) and clone ID PBX0143E06 had no GenBank entry.

The microarray chip contained 2 alpha-tubulin genes, 3 ras-related proteins and a phosphatase 2A regulatory subunit. The mean of 4 data points of alpha-tubulin, a house keeping gene, is 0.85 using NF from landmarks suggesting minimal change after treatment at stage 8. Among the 3 ras-related protein genes, RAB-1A was up-regulated (2.10) and RAP-1B and RAB-9 were down-regulated by PMA treatment at stage 8. Phosphatase 2A regulatory subunit was up-regulated by PMA treatment ˜3-fold at stage 8 (see PBX0140D01 in Table 1, Panel A second from last entry).

Microarray analysis of PMA-treatment of Xenopus laevis cleavage stage embryos compared to untreated embryos identified 25 Xenopus genes that were highly up-regulated (3-8 fold; Table 1, Panel A) and 24 genes that were highly down-regulated (0.6-0.15 fold; Table 3).

### Example 2

Identification of Genes that are Similarly or Differentially Regulated by PMA-Treatment in Cleavage and Neurulation Stage Embryos.

Comparing the data generated by microarray analysis of gene expression using untreated and PMA-treated cleavage or neurulation stage embryos allows for identification of Xenopus genes that are similarly or differentially regulated by PMA during distinct periods of embryogenesis. Genes corresponding to ESTs PBX0134G08, PBX0144C12, PBX0136C09, PBX0134H03, PBX0136F03, PBX0143E06 and PBX0137G06 were highly up-regulated, and PBX0144A09 was highly down-regulated, by PMA-treatment of cleavage and neurulation stage embryos. Genes corresponding to ESTs PBX0134C10, PBX0139B06, PBX0144E04, PBX0137A11, PBX0134G10, PBX0141G10, PBX0134E09, PBX0138A04, PBX0134E04, PBX0145A06, PBX0138D01, PBX0135C06, PBX0142E09, PBX0139A08, PBX0134G11, PBX0145H10, PBX0140DO01 and PBX0135A08 were highly up-regulated by PMA-treatment of the cleavage stage embryo but not the neurulation stage embryo.

Throughout the application, various publications, including United States patents, are referenced by author and year and patents by number. Full citations for the publications are listed below. The disclosures of the publications and patents in their entireties are hereby incorporated by reference into the application in order to more fully describe the state of the art to which the present invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

TABLE 1
Panel A. Up-regulated genes after PMA treatment of Xenopus embryos at cleavage stage.
Ratios were calculated for each spot by dividing the spot volume (integrated intensity minus background)
of the Cy5 channel (red, 635 nm) with the spot volume of the Cy3 (green, 532 nm). Medians were used to
calculate ratios for the table. All ratios were then multiplied by normalization factor (NF) to correct for
differences in labeling efficiency between probes

| | | | | Normalized | | |
|---|---|---|---|---|---|---|
| | Ratio of Medians (Cy5/Cy3) | | | by all Data Points | by Landmarks | Block Number |
| Clone ID | First Spot | Second Spot | Mean | (1.28) | (1.59) | on the Chips |
| PBX | 3.33 | 6.67 | 5.00 ± 1.67^{a} | 6.40 | 7.95 | 5 |
| 0134C10 | | | | | | |
| PBX | 4.25 | 5.33 | 4.79 ± 0.54 | 6.13 | 7.62 | 15 |
| 0134G08 | | | | | | |
| PBX | 4.75 | 2.23 | 3.49 ± 1.26 | 4.47 | 5.55 | 7 |
| 0144C12 | | | | | | |
| PBX | 3.80 | 3.17 | 3.49 ± 0.31 | 4.47 | 5.55 | 5 |
| 0136C09 | | | | | | |
| PBX | 3.67 | 3.17 | 3.42 ± 0.25 | 4.38 | 5.44 | 14 |
| 0134H03 | | | | | | |
| PBX | 4.00 | 2.86 | 3.43 ± 0.57 | 4.39 | 5.45 | 20 |
| 0139B06 | | | | | | |
| PBX | 3.67 | 3.17 | 3.42 ± 0.25 | 4.38 | 5.45 | 5 |
| 0144E04 | | | | | | |
| PBX | 2.17 | 4.50 | 3.34 ± 1.66 | 4.28 | 5.31 | 17 |
| 0137A11 | | | | | | |
| PBX | 3.50 | 3.11 | 3.31 ± 0.20 | 4.24 | 5.26 | 10 |
| 0136F03 | | | | | | |
| PBX | 2.71 | 3.83 | 3.27 ± 0.56 | 4.19 | 5.20 | 15 |
| 0134G10 | | | | | | |
| PBX | 2.53 | 3.72 | 3.13 ± 0.59 | 4.01 | 4.98 | 31 |
| 0141G10 | | | | | | |
| 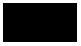 | 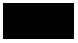 | 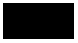 | 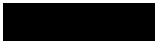 | 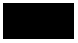 | 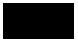 | 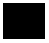 |
| 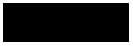 | | | | | | |
| PBX | 1.64 | 4.33 | 2.99 ± 1.32 | 3.83 | 4.75 | 3 |
| 0138A04 | | | | | | |
| PBX | | | | | | |
| 0134E04 | 2.75 | 3.00 | 2.88 ± 0.12 | 3.69 | 4.58 | 11 |
| PBX | 2.27 | 3.44 | 2.86 ± 0.59 | 3.66 | 4.55 | 19 |
| 0145A06 | | | | | | |
| PBX | 3.50 | 2.10 | 2.80 ± 0.70 | 3.58 | 4.45 | 6 |
| 0138D01 | | | | | | |
| PBX | 3.00 | 2.40 | 2.70 ± 0.30 | 3.46 | 4.29 | 23 |
| 0135C06 | | | | | | |
| PBX | 2.50 | 2.93 | 2.72 ± 0.21 | 3.48 | 4.32 | 9 |
| 0142E09 | | | | | | |
| PBX | 2.00 | 3.29 | 2.65 ± 0.65 | 3.39 | 4.21 | 19 |
| 0139A08 | | | | | | |
| PBX | 2.50 | 2.40 | 2.45 ± 0.05 | 3.14 | 3.90 | 13 |
| 0134G11 | | | | | | |
| PBX | 2.92 | 1.91 | 2.42 ± 0.50 | 3.10 | 3.85 | 27 |
| 0143E06 | | | | | | |
| PBX | 2.90 | 1.86 | 2.38 ± 0.52 | 3.05 | 3.78 | 32 |
| 0145H10 | | | | | | |
| PBX | 2.22 | 2.47 | 2.35 ± 0.13 | 3.01 | 3.74 | 31 |
| 0137G06 | | | | | | |
| PBX | 2.09 | 2.50 | 2.30 ± 0.20 | 2.94 | 3.66 | 6 |
| 0140D01 | | | | | | |
| 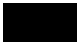 | 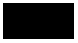 | 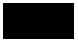 | 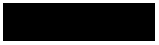 | 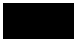 | 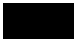 | 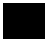 |
| 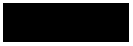 | | | | | | |

^{a}difference between value of a spot and mean value

TABLE 1
Panel B. Identification of up-regulated genes at cleavage stage with the NIEHS EST nucleotide sequence entry
size in GenBank bigger than 500 bp. Xenonus aldolase sequence matched with both PBX0141G10 and
PBX0145H10 (bold type).

| Clone ID, Location in chip, GenBank No., | BLAST results using sequence of the up-regulated gene |
|---|---|
| PBX0141G10, H3c8/H3h8, AW644589, 590 bp | D38621 Xenopus aldolase, 118/134 (88%) |
| | M75873 Xenopus elongation factor 1-alpha-o, 116/133 (87%) |
| | X53846 Xenopus elongation factor 1-alpha-o, 67/78 (85%) |
| | M67485 Xenopus elongation factor 1-alpha-o, 67/78 (85%) |
| | Y13284 Xenopus fibronectin, 61/65 (93%) |
| | X04807 Xenopus Stage-specific epidermal type I keratin B2 |
| | (embryo- and larval-specific), 70/83 (84%) |
| | M99581 Xenopus gamma-crystallin (gcry3), 49/55 (89%) |
| PBX0134E09, C1b4/C1h4, AW643934, 556 bp: | no match in other GenBank entry |
| PBX0143E06,G3d6/G3f6 | no sequence data in GenBank |
| PBX0145H10, H4e2/h4e8, AW644919, 617 bp | U23535 Xenopus epithelial sodium channel, |
| | alpha subunit,397/468 (84%) |
| | X05025 Xenopus ribosomal protein I14, 322/377 (85%) |
| | M22984 Xenopus oocytes poly(A) RNA that hybridizes to |
| | a cloned interspersed repeat, 220/251 (87%) |
| | D38621, Xenopus aldolase, 201/239 (84%) |
| | X71081, Xenopus ribosomal protein S8, 96/105 (91%) |
| | Z54313 Xenopus borealis U7 snRNA genes, 232/282 (82%) |
| PBX0137G06, H3a8/H3i8, AW644223, 668 bp: | no match in other GenBank entry. |
| PBX0135A08, E3a9, AW643975, 549 bp | AB032767,chicken, CD9antigen,123/152 (80%) |
| | BC011988 human, similar to CD9 antigen, 122/154 (79%) |

TABLE 2
Comparison of % increase of mRNA expression after
PMA treatment at cleavage stage obtained by microarray
analysis with results by RT-PCR.

| | by Microarray analysis | | | |
|---|---|---|---|---|
| | (NF by all data point) | | | by RT-PCR |
| Gene | Stage 8 | Stage 15 | Stage 8 | Stage 15 |
| PBX0135A08 | 270%^{a} | no change | 260% | no change |
| PBX013409 | 380% | no change | highly | no change |
| | | | induced^{b} | |

^{a}% of control.
^{b}not detected in control but strong band after PMA treatment.

TABLE 3
Down-regulated genes after PMA treatment of Xenopus embryos
at cleavage stage. Ratios were calculated for each spot by
dividing the spot volume (integrated intensity minus
background) of the Cy5 channel (red, 635 nm) with the spot
volume of the Cy3 (green, 532 nm). Both mean and median
values were calculated. Medians were used to calculate ratios
for the table. All ratios were then multiplied by
normalization factor (NF).

| | Ratio of | | | Normalization | | (CY3/CY5) | | |
|---|---|---|---|---|---|---|---|---|
| | Medians | | | by all | | by all | | Block |
| | (Cy5/Cy3) | | | Data | by | Data | by | Number |
| | First | Second | | Points | Landmarks | Points | Landmarks | on the |
| Clone ID | Spot | Spot | Mean | (1.3) | (1.6) | (1.3) | (1.6) | Chip |
| PBX 0144A09 | 0.06 | 0.09 | 0.07 ± 0.02^{a} | 0.10 | 0.15 | 10.33 | 6.45 | 1 |
| PBX 0145E05 | 0.11 | 0.07 | 0.09 ± 0.02 | 0.12 | 0.18 | 8.69 | 5.43 | 25 |
| PBX 0138C12 | 0.14 | 0.13 | 0.13 ± 0.01 | 0.17 | 0.28 | 5.74 | 3.59 | 7 |
| PBX 0141F12 | 0.10 | 0.17 | 0.14 ± 0.03 | 0.18 | 0.28 | 5.70 | 3.56 | 28 |
| PBX 0140H03 | 0.09 | 0.19 | 0.14 ± 0.05 | 0.18 | 0.28 | 5.64 | 3.52 | 14 |
| PBX 0139F05 | 0.15 | 0.12 | 0.14 ± 0.02 | 0.18 | 0.28 | 5.64 | 3.52 | 26 |
| PBX 0138G10 | 0.10 | 0.18 | 0.14 ± 0.04 | 0.19 | 0.30 | 5.36 | 3.35 | 15 |
| PBX 0139D03 | 0.04 | 0.26 | 0.15 ± 0.11 | 0.20 | 0.32 | 5.04 | 3.15 | 24 |
| PBX 0145E10 | 0.21 | 0.12 | 0.17 ± 0.05 | 0.22 | 0.35 | 4.57 | 2.85 | 27 |
| PBX 0145E09 | 0.16 | 0.19 | 0.17 ± 0.02 | 0.23 | 0.36 | 4.42 | 2.76 | 25 |
| PBX 0136B04 | 0.11 | 0.24 | 0.17 ± 0.06 | 0.23 | 0.36 | 4.41 | 2.76 | 4 |
| PBX 0138E10 | 0.15 | 0.21 | 0.18 ± 0.03 | 0.23 | 0.37 | 4.29 | 2.68 | 11 |
| PBX 0138G12 | 0.17 | 0.21 | 0.19 ± 0.02 | 0.24 | 0.39 | 4.09 | 2.56 | 15 |
| PBX 0142E01 | 0.14 | 0.22 | 0.18 ± 0.04 | 0.24 | 0.38 | 4.23 | 2.64 | 9 |
| PBX 0139F12 | 0.20 | 0.16 | 0.18 ± 0.02 | 0.24 | 0.38 | 4.21 | 2.63 | 28 |
| PBX 0143C01 | 0.16 | 0.22 | 0.19 ± 0.03 | 0.25 | 0.39 | 4.07 | 2.54 | 21 |
| PBX 0142H06 | 0.15 | 0.23 | 0.19 ± 0.04 | 0.25 | 0.40 | 4.04 | 2.52 | 16 |
| PBX 0140F01 | 0.24 | 0.16 | 0.20 ± 0.04 | 0.26 | 0.42 | 3.83 | 2.39 | 10 |
| PBX 0139H03 | 0.26 | 0.20 | 0.23 ± 0.03 | 0.30 | 0.47 | 3.38 | 2.11 | 30 |
| PBX 0145A10 | 0.28 | 0.22 | 0.25 ± 0.03 | 0.32 | 0.52 | 3.10 | 1.93 | 19 |
| PBX 0137E10 | 0.31 | 0.21 | 0.26 ± 0.05 | 0.34 | 0.54 | 2.96 | 1.85 | 27 |
| PBX 0141B03 | 0.38 | 0.18 | 0.28 ± 0.10 | 0.36 | 0.58 | 2.78 | 1.74 | 18 |
| PBX 0141G06 | 0.17 | 0.41 | 0.29 ± 0.12 | 0.38 | 0.60 | 2.67 | 1.67 | 31 |
| PBX 0143D10 | 0.22 | 0.40 | 0.31 ± 0.11 | 0.40 | 0.64 | 2.51 | 1.57 | 32 |

^{a}difference between value of a spot and mean value

TABLE 4
Up-regulated genes after PMA treatment of Xenopus embryos
at neurulation stage. Ratios were calculated for each spot by
dividing the spot volume (integrated intensity minus background)
of the Cy5 channel (red, 635 nm) with the spot volume of the
Cy3 (green, 532 nm). Both mean and median values were
calculated. Medians were used to calculate ratios for the table.
All ratios were then multiplied by normalization factor (NF).

| | Ratio of | | | | | |
|---|---|---|---|---|---|---|
| | Medians | | | Normalized | | |
| | (Cy5/Cy3) | | | | by | Block |
| | First | Second | | by all Data | Landmarks | Number |
| Clone ID | Spot | Spot | Mean | Points (0.469) | (0.25) | On the Chip |
| PBX 0143E06 | 6.04 | 6.78 | 6.41 ± 0.3^{a} | 3.01 | 1.60 | 27 |
| PBX 0138E11 | 5.02 | 7.56 | 6.29 ± 1.27^{a} | 2.95 | 1.57 | 9 |
| PBX 0136F03 | 3.67 | 8.68 | 6.18 ± 2.5^{a} | 2.90 | 1.54 | 10 |
| PBX 0136C09 | 4.19 | 8.05 | 6.12 ± 1.93^{a} | 2.87 | 1.53 | 5 |
| PBX 0136C12 | 5.29 | 6.75 | 6.02 ± 0.73^{a} | 2.82 | 1.51 | 7 |
| PBX 0138H03 | 6.36 | 5.14 | 5.75 ± 0.61^{a} | 2.70 | 1.44 | 14 |
| PBX 0143E11 | 4.62 | 6.65 | 5.64 ± 1.01^{a} | 2.64 | 1.41 | 25 |
| PBX 0141H12 | 7.46 | 3.75 | 5.61 ± 1.85^{a} | 2.63 | 1.40 | 32 |
| PBX 0136G08 | 4.48 | 6.37 | 5.43 ± 0.94^{a} | 2.54 | 1.36 | 15 |
| PBX 0136G06 | 4.95 | 5.89 | 5.42 ± 0.47^{a} | 2.54 | 1.36 | 15 |

^{a}difference between value of a spot and mean value

TABLE 5
Down-regulated genes after PMA treatment of Xenopus embryos
at neurulation stage. Ratios were calculated for each spot by
dividing the spot volume (integrated intensity minus
background) of the Cy5 channel (red, 635 nm) with the spot
volume of the Cy3 (green, 532 nm). Both mean and median
values were calculated. Medians were used to calculate ratios
for the table. All ratios were then multiplied by
normalization factor (NF).

| | Ratio of | | | Normalization | | (CY3/CY5) | | |
|---|---|---|---|---|---|---|---|---|
| | Medians | | | by all | | by all | | Block |
| | (Cy5/Cy3) | | | Data | by | Data | by | Number |
| | First | Second | | Points | Landmarks | Points | Landmarks | on the |
| Clone ID | Spot | Spot | Mean | (0.469) | (0.25) | (0.469) | (0.25) | Chip |
| PBX 0141H05 | 0.73 | 0.015 | 0.37 ± 0.36^{a} | 0.174 | 0.093 | 5.76 | 10.8 | 30 |
| PBX 0135H08 | 0.78 | 0.84 | 0.81 ± 0.03^{a} | 0.380 | 0.203 | 2.63 | 4.93 | 32 |
| PBX 0145D04 | 0.9 | 0.95 | 0.92 ± 0.02^{a} | 0.431 | 0.230 | 2.32 | 4.35 | 24 |
| PBX 0140A10 | 0.89 | 1.00 | 0.95 ± 0.06^{a} | 0.446 | 0.238 | 2.24 | 4.20 | 3 |
| PBX 0145D06 | 0.98 | 1.00 | 0.99 ± 0.01^{a} | 0.464 | 0.248 | 2.15 | 4.03 | 24 |
| PBX 0144A09 | 0.83 | 1.16 | 1.00 ± 0.17^{a} | 0.469 | 0.250 | 2.13 | 4.00 | 1 |
| PBX 0135G07 | 0.98 | 1.04 | 1.01 ± 0.03^{a} | 0.474 | 0.253 | 2.11 | 3.95 | 29 |
| PBX 0141B10 | 0.92 | 1.12 | 1.02 ± 0.10^{a} | 0.478 | 0.255 | 2.09 | 3.92 | 20 |
| PBX 0139G06 | 1.06 | 1.06 | 1.06 ± 0.00^{a} | 0.497 | 0.265 | 2.01 | 3.77 | 31 |
| PBX 0137F04 | 0.95 | 1.21 | 1.08 ± 0.13^{a} | 0.507 | 0.270 | 1.97 | 3.70 | 28 |

^{a}difference between value of a spot and mean value

### REFERENCES

1. Bantel J A, Finch, R A, Fort, D J, Stover, E L, Hull, M, Kunsher-King, M, and A M Gaudet-Hull (1999) Phase III interlaboratory study of FETAX. Part 3. FETAX validation using 12 compounds with and without an exogenous metabolic activation system. J. Appl. Toxicol. 19(6): 447-472.

2. Fort D J, Stover, E L, Farmer, D R, and J K Leman (2000) Assessing the predictive validity of frog embryo teratogenesis assay—Xenopus (FETAX). Teratog: Carcinog. Mutagen. 20(2): 87-98.

3. Newport J and M. Kirschner (1982a) A major developmental transition in early Xenopus embryos: I. Characterization and timing of cellular changes at the midblastula transition. Cell 30: 675-686.

4. Newport J and M. Kirschner (1982b) A major developmental transition in early Xenopus embryos: II. Control of the onset of transcription. Cell 30: 687-696.

5. Nakakura, N, Miura, T, Yamana, K, Ito, A, and K Shiokawa (1987) Synthesis of heterogeneous mRNA-like RNA and low-molecular-weight RNA before the midblastula transition in embryos of Xenopus laevis. Dev. Biol. 123: 421-429.

6. Adams, M. D., Kelley, J. M., Gocayne, J. D., Dubnick, M., Polymeropoulos, M. H., Xiao, H., Merril, C. R., Wu, A., Olde, B., Moreno, R. F., Kerlavage, A. R., McCombie, W. R. and Venter, J. C. (1991) Complimentary DNA sequencing: Expressed sequence tags and human genome project. Science 252:1651-1656.

7. Schena, M., Shalon, D., Davis, R. W. and Brown, P. O. (1995) Quantitative monitoring of gene expression patterns with a complimentary DNA microarray. Science 270:467-487.

8. Schena, M., Shalon, D., Heller, R. Chai, A., Brown, P. O. (1996) Parallel human genome analysis: Microarray-based expressions monitoring of 1000 genes. Proc. Natl. Acad. Sci. USA 93:10614-19.

9. Zhang, M. Q. Genome Res. 9:681-688 (1999) (http:/www.genome.org/cgi/content/full/9/8/681).

10. Nuwaysir, E. F., Bittner, M., Trent, J., Barrett, J. C., Afshari, C. A. (1999) Microarrays and toxicology: The advent of toxicogenomics. Mol. Carcinogen. 24:153-159.

11. Altmann, C. R., Bell, E., Sczyrba, A., Pun, J., Bekiranov, S., Gaasterland, T. and Brivanlou, A. H. (2001) Microarray-based analysis of early development in Xenopus laevis. Dev. Biol. 236:64-75.

12. Nieuwkoop, P. D. and Faber. J. (1967) Normal Table of Xenopus laevis (Daudin) N. Holland in chapter VII, pp. 162-188.

What is claimed is:

1. A screen for detecting affects of chemicals on gene expression comprising animal cleavage stage embryos and detecting means for detecting changes in gene expression.

2. The screen according to claim 1, wherein said embryos are vertebrate embryos.

3. The screen according to claim 2, wherein said embryos are embryos from aquatic species.

4. The screen according to claim 3, wherein said embryos are amphibian.

5. The screen according to claim 4, wherein said embryos are Xenopus.

6. The screen according to claim 5, wherein said embryos are Xenopus laevis.

7. A screen for identifying and characterizing chemicals as toxicants based on the affect of the chemical on gene expression, said screen comprising animal cleavage stage embryos.

8. The screen according to claim 7, wherein said embryos are vertebrate embryos.

9. The screen according to claim 8, wherein said embryos are embryos from aquatic species.

10. The screen according to claim 9, wherein said embryos are amphibian.

11. The screen according to claim 10, wherein said embryos are Xenopus.

12. The screen according to claim 11, wherein said embryos are Xenopus laevis.

13. The screen according to claim 7, wherein the chemicals to be tested are inducers of cellular proliferation.

14. The screen according to claim 13, wherein said inducers are phorbol esters.

15. The screen according to claim 14, wherein said phorbol ester is phorbol 12-myristate 13-acetate.

16. A microarray screen for detecting and measuring the affects of chemicals on gene expression in animal cleavage stage embryos.

17. The microarray screen according to claim 16, wherein said embryos are vertebrate embryos.

18. The microarray screen according to claim 17, wherein said embryos are embryos from aquatic species.

19. The microarray screen according to claim 18, wherein said embryos are amphibian.

20. The microarray screen according to claim 19, wherein said embryos are Xenopus.

21. The microarray screen according to claim 20, wherein said embryos are Xenopus laevis.

22. Markers of chemical exposure identified using the screen according to claim 1.

23. Markers of chemical exposure identified using the screen according to claim 1 as listed in Table 1, Panel A, and Table 3 and corresponding genes in other species

24. Markers of teratogenesis identified using the screen according to claim 1.

25. Markers of teratogenesis identified using the screen according to claim 1 as listed in Table 1, Panel A, and Table 3 and corresponding genes in other species.

26. A screen for identifying and characterizing chemicals as toxicants based on the affect of the chemical on gene expression, said screen comprising animal embryos undergoing cleavage and neurulation.

27. The screen according to claim 26, wherein said embryos are vertebrate embryos.

28. The screen according to claim 27, wherein said embryos are embryos from aquatic species.

29. The screen according to claim 28, wherein said embryos are amphibian.

30. The screen according to claim 29, wherein said embryos are Xenopus.

31. The screen according to claim 30, wherein said embryos are Xenopus laevis.

32. A treatment enabling the transfer of biotinylated DNA to a membrane following gel electrophoresis, said treatment including the steps of:
depurinating the DNA; and
denaturing the DNA.

33. A treatment enabling the transfer of biotinylated PCR products to a membrane following gel electrophoresis, said treatment including the steps of:
depurinating the PCR products; and
denaturing the PCR products.

34. A treatment enabling the transfer of biotinylated PCR products obtained by reverse-transcription of mRNA to a membrane following gel electrophoresis, said treatment including the steps of:
depurinating the PCR products; and
denaturing the PCR products.
